(19) United States
(12) Patent Application Publication
(10) Pub. No.: US 2023/0121814 A1
(43) Pub. Date: Apr. 20, 2023

(54) SYSTEMS AND METHODS FOR QUANTIFYING TEAM PERFORMANCE CAPACITY

(71) Applicant: Veritas Index Institute, Wilmington, DE (US)

(72) Inventor: Steven Lam, Lexington, MA (US)

(21) Appl. No.: 17/975,098

(22) Filed: Oct. 27, 2022

Related U.S. Application Data
(63) Continuation-in-part of application No. 17/857,825, filed on Jul. 5, 2022.
(60) Provisional application No. 63/218,497, filed on Jul. 6, 2021.

(51) Int. Cl. G06Q 10/0631; G16H 40/67

(52) U.S. Cl. CPC G06Q 10/063114; G16H 40/67

(57) ABSTRACT

This disclosure is related to quantifying team capacity. Active lifestyle factor data, passive lifestyle factor data, physiological data input, external conditions data, and emotional intelligence (EI) score data associated with at least one user in a team at a first time may be used to compute a first performance capacity score for the at least one user. Active lifestyle factor data, passive lifestyle factor data, physiological data input, external conditions data, and emotional intelligence (EI) score data associated with at least one user in the team at a second time may be used to compute a second performance capacity score for the at least one user. An aggregated performance capacity score for at least one user on the team based on the first performance capacity score and the second performance capacity score. A team performance score may be computed based on individual role data associated with at least one user.

### CROSS-REFERENCE TO RELATED APPLICATIONS

This application is a continuation-in-part application of U.S. Non-Provisional Pat. Application No.: 17/857,825, filed Jul. 05, 2022, titled “SYSTEMS AND METHODS FOR MEASURING PERFORMANCE.” Both applications claim priority to and benefit of U.S. Provisional Application 63/218,497, filed Jul. 06, 2021, titled “SYSTEMS AND METHODS FOR PERFORMANCE ENHANCEMENT RECOMMENDATIONS AND CANDIDATE RANKING.” Both applications are herein incorporated by reference in their entirety.

### BACKGROUND

### Field of the Art

The systems and methods disclosed herein are related generally to performance measurement and team performance capacity measurement.

### Discussion of the State of the Art

Recently, telemedicine and content-streaming exercise devices have made remote personal care more accessible. Executive Function diagnoses and tracking which were previously only available at medical facilities and university laboratories are now available at home. However, data from a single source and/or session in time may be inaccurate, misleading, or may not tell the entire story about an individual’s performance. Currently, there are no tools that enable continuous detection of a user’s executive function.

It is common for people to work in teams to achieve goals. Each member of a team may have a capacity to perform a task. As a team member feels burnt out, their performance capacity may suffer. The capacity to perform a task may be influenced by internal factors, such as, for example, a change in weather, external factors, such as, for example, a heart rate, or a combination of the two, such as, for example, how long after an alarm sounds a team member gets up. As more members of a team begin to feel burnt out or essential members of a team begin to feel burnt out, it may become more uncertain that the team can achieve its goals. What is needed are systems and methods for measuring and quantifying team performance capacity.

### SUMMARY

Disclosed herein are computer implemented methods for quantifying individual executive function performance measures and based on mental status data, lifestyle data and biofeedback data. An example computer implemented method may comprise obtaining, via at least one first user worn sensor, first physiology data associated with the user. The example computer implemented method may comprise obtaining, via at least one first user device, engagement factor data. The engagement factor data may comprise user input associated with at least one of punctuality, sleep, diet, waste excretion, exercise, and responses to mental status questionnaires. The example computer implemented method may comprise converting, via a processor, the first physiology data and the engagement factor data into lifestyle factor data having a standard format. The example computer implemented method may comprise generating, via a processor, a first user score associated with the lifestyle factor data. The example computer implemented method may comprise generating, via a processor, mental status data based on a user’s responses to a mental status questionnaires. The example computer implemented method may comprise obtaining, via a processor, executive function performance outcome data associated with a task performed by the user under at least one condition. The at least one condition may comprise at least one of a baseline condition, a mental stimulation condition, and a physical exertion condition. The example computer implemented method may comprise obtaining, via the at least one first user worn sensor, second physiology data associated with the task performed by the user under the at least one condition. The example computer implemented method may comprise converting, via a processor, the executive function performance outcome data into performance factor data having a standard format. The example computer implemented method may comprise converting, via a processor, the second physiology data into biometric factor data having a standard format. The example computer implemented method may comprise converting, via a processor, mental questionnaire data into mental factor data having a standard format. The example computer implemented method may comprise generating, via a processor, a second user score associated with the biometric factor data and performance factor data. The example computer implemented method may comprise generating, via a processor, a third user score based on the first user score and the second user score.

The Mental status questionnaire data may comprise a plurality of components, each component associated with 5 major aspects of emotional intelligence: Self Awareness, Self Regulation, Motivation, Adaptability and Interpersonal.

The first physiology data may comprise at least one of heart rate (HR) data, heart rate variability (HRV) data, sleep time data, wake time data, sleep duration data, deep sleep duration data, rapid eye movement (REM) sleep duration data, and calories burned data.

The lifestyle factor data may comprise a plurality of components. Each component may be associated with at least one of punctuality, sleep, diet, exercise, waste excretion, and mental status.

The lifestyle factor data may comprise at least one of active lifestyle factor data and passive lifestyle factor data.

The example computer implemented method may comprise categorizing each lifestyle factor as associated with toxicity generation or toxicity reduction. The example computer implemented method may comprise computing a net toxicity metric from the lifestyle factor data.

The first user score may provide an indication of user performance potential.

The performance potential may be determined as a function of at least one of toxicity accumulation over time and toxicity reduction over time.

The second physiology data may comprise at least one of heart rate (HR) data, heart rate variability (HRV) data, blood pressure data, posture data, and hormonal data.

The mental stress condition may be invoked by applying at least one external stimuli to be sensed by the user prior to or during performing the task.

The physical exertion condition may be invoked by requiring the user to perform a physical activity during and/or prior to or during performing the task.

The physical exertion condition may comprise requiring the user to achieve certain physiological criteria prior to or during performing the task.

Biometric change data may be computed. The biometric change data indicating a change in the second physiology data associated with at least one of the mental stress condition and the physical stress condition as compared to the first physiology data associated with the baseline condition.

The second user score may indicate at least one of an average biometric change associated with a plurality of conditions and an average performance change associated with a plurality of conditions.

The third user score may comprise an adjustment of the second user score based on a ratio of the first user score relative to a first user score threshold target.

The first user score threshold target may be indicative of a threshold below which performance potential is reduced.

Disclosed herein are computer systems for quantifying individual executive function performance measures and based on lifestyle data and biofeedback data. An example computing system may comprise at least one computing processor. The example computing system may comprise memory. The memory may comprise instructions. The instructions, when executed by the at least one computing processor, may enable the computing system to obtain, via at least one first user worn sensor, first physiology data associated with the user. The instructions, when executed by the at least one computing processor, may enable the computing system to obtain, via at least one first user device, engagement factor data. The engagement factor data may comprise user input associated with at least one of sleep, diet, waste excretion, exercise, and mental status. The instructions, when executed by the at least one computing processor, may enable the computing system to convert, via a processor, the first physiology data and the engagement factor data into lifestyle factor data having a standard format. The instructions, when executed by the at least one computing processor, may enable the computing system to generate, via a processor, a first user score associated with the lifestyle factor data. The instructions, when executed by the at least one computing processor, may enable the computing system to obtain, via a processor, performance outcome data associated with a task performed by the user under at least one condition. The at least one condition may comprise at least one of a baseline condition, a mental stimulation condition, and a physical exertion condition. The instructions, when executed by the at least one computing processor, may enable the computing system to obtain, via the at least one first user worn sensor, second physiology data associated with the task performed by the user under the at least one condition. The instructions, when executed by the at least one computing processor, may enable the computing system to convert, via a processor, the performance outcome data into performance factor data having a standard format. The instructions, when executed by the at least one computing processor, may enable the computing system to convert, via a processor, the second physiology data into biometric factor data having a standard format. The instructions, when executed by the at least one computing processor, may enable the computing system to generate, via a processor, a second user score associated with the biometric factor data and performance factor data. The instructions, when executed by the at least one computing processor, may enable the computing system to generate, via a processor, a third user score based on the first user score and the second user score.

The first physiology data may comprise at least one of heart rate (HR) data, heart rate variability (HRV) data, sleep time data, wake time data, sleep duration data, rapid eye movement (REM) sleep duration data, deep sleep duration data, and calories burned data.

Disclosed herein are on-transitory computer readable medium comprising instructions. When executed by a processor, the instructions may enable the processor to obtain, via at least one first user worn sensor, first physiology data associated with the user. When executed by a processor, the instructions may enable the processor to obtain, via at least one first user device, engagement factor data. The engagement factor data may comprise user input associated with at least one of sleep, diet, waste excretion, exercise, and mental status. When executed by a processor, the instructions may enable the processor to convert, via a processor, the first physiology data and the engagement factor data into lifestyle factor data having a standard format. When executed by a processor, the instructions may enable the processor to generate, via a processor, a first user score associated with the lifestyle factor data. When executed by a processor, the instructions may enable the processor to obtain, via a processor, performance outcome data associated with a task performed by the user under at least one condition. The at least one condition may comprise at least one of a baseline condition, a mental stimulation condition, and a physical exertion condition. When executed by a processor, the instructions may enable the processor to obtain, via the at least one first user worn sensor, second physiology data associated with the task performed by the user under the at least one condition. When executed by a processor, the instructions may enable the processor to convert, via a processor, the performance outcome data into performance factor data having a standard format. When executed by a processor, the instructions may enable the processor to convert, via a processor, the second physiology data into biometric factor data having a standard format. When executed by a processor, the instructions may enable the processor to generate, via a processor, a second user score associated with the biometric factor data and performance factor data. When executed by a processor, the instructions may enable the processor to generate, via a processor, a third user score based on the first user score and the second user score.

The first physiology data may comprise at least one of heart rate (HR) data, heart rate variability (HRV) data, sleep time data, wake time data, sleep duration data, rapid eye movement (REM) sleep duration data, deep sleep duration, and calories burned data.

The lifestyle factor data may comprise a plurality of components, each component associated with at least one of punctuality, sleep, diet, exercise, waste excretion, and mental status.

The Mental status questionnaire data may comprise a plurality of components, each component associated with 5 major aspect of emotional intelligence: Self Awareness, Self Regulation, Motivation, Adaptability and Interpersonal.

Disclosed herein are computer implemented methods for quantifying performance capacity associated with a team. An example method may comprise obtaining, via at least one of a first user worn sensor and a first user device, active lifestyle factor data associated with at least one user in the team. The example method may comprise obtaining, via the first user worn sensor, passive lifestyle factor data associated with at least one user in the team. The example method may comprise obtaining, via the first user device, physiological data input by at least one user in a team. The example method may comprise obtaining, via an application programming interface (API), external conditions data. The example method may comprise computing emotional intelligence (EI) score data based on EI input data. The example method may comprise computing a first performance capacity score for at least one user in the team, based on the active lifestyle data, the external data, and user input physiological data. The example method may comprise computing a second performance capacity score for at least one user in the team based on active lifestyle data, passive lifestyle data, and API data. The example method may comprise computing an aggregated performance capacity score for at least one user on the team based on the first performance capacity score and the second performance capacity score. The example method may comprise computing a team performance score based on individual role data.

Active lifestyle factor data may comprise at least one of exercise data, dinnertime data, bedtime data, and punctuality data.

Exercise data may comprise at least one of exercise duration data and exercise intensity data.

Punctuality data may comprise at least one of calendar event data and user performance of calendar event data.

Passive lifestyle data may comprise at least one of resting heart rate data, REM sleep duration data, and deep sleep duration data.

User input physiological data may comprise food intake data.

External conditions data may comprise at least one of outdoor temperature data, dew point data, outdoor temperature data, atmospheric pressure data, and Earth to moon distance data.

The EI input data may be input on the first user device by at least one user in the team.

The example method may comprise providing remote access to users over a network so any one of the users can update information about the physiological data and the EI input data.

Any one of the users may provide the update information in a non-standardized format depending on at least one of the hardware and platform used by the any one of the users.

The example method may comprise converting the obtained passive lifestyle factor data, the physiological data, the API data, the EI score data, and the non-standardized update information into a standardized format.

The first performance capacity score may be computed in part based on a relationship between the active lifestyle data and the API data.

The first performance capacity score may be based in part based on a relationship between bedtime data and sunset data.

The second performance capacity score may be based on at least one of an exercise measure, a resting heart rate measure, a relationship between dinner time and sunset, a sleep quality measure, and a relationship between bedtime data and sunset data.

The role data may indicate an individual’s contribution to the team.

The role data may comprise at least one of a position, hours worked, and relative salary.

The relative salary may be a ratio representing an associated user’s salary compared to the total team salary.

The example method may comprise automatically generating a message containing an aggregated performance capacity score and the team performance score whenever new lifestyle factor data, passive lifestyle data, physiological data, external condition data, EI input data, or non-standardized update data is obtained.

The example method may comprise transmitting the message to a plurality of users over the computer network in near real time, so that the plurality of users have near immediate access to at least one of an aggregated performance capacity score and the team performance score.

The team performance score may be further based on a third performance capacity score associated with another user.

The present invention solves the technical problem of computing a performance capability score. Currently, the underlying data is difficult to obtain, and if it is obtained, it is difficult to standardize the data to create meaningful inferences on top of the data. Generally, all of this data may be obtained for a plurality of devices, each with differing data gathering and transmission capability, and stored in different data formats. Additionally, even if the underlying data is standardized, it is technically challenging to make real time assessments to that data and enable multiple stakeholders to draw insights and recommendations from the data on their various computing devices.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a system for evaluating pressure performance in accordance with an exemplary embodiment of the invention.

FIG. 2 illustrates a system for evaluating pressure performance in accordance with an exemplary embodiment of the present invention.

FIG. 3 illustrates an exemplary process for evaluating pressure performance according to one embodiment of the invention.

FIG. 4 illustrates one embodiment of the computing architecture that supports an embodiment of the inventive disclosure.

FIG. 5 illustrates components of a system architecture that supports an embodiment of the inventive disclosure.

FIG. 6 illustrates components of a computing device that supports an embodiment of the inventive disclosure.

FIG. 7 illustrates components of a computing device that supports an embodiment of the inventive disclosure.

FIG. 8 illustrates a system for quantifying team performance capacity in accordance with an exemplary embodiment of the present invention.

FIG. 9 illustrates an exemplary process for quantifying team performance capacity according to one embodiment of the invention.

### DETAILED DESCRIPTION

The inventive systems and methods (hereinafter sometimes referred to more simply as “system” or “method”) described herein evaluate intrinsic executive function. Specifically, the inventive systems and methods allow a user to evaluate one’s executive function under pressure. The inventive systems and methods may use a first set of mental status data to determine baseline user data. The inventive systems and methods may use user mental data and determined baseline user mental data to determine performance potential for the user for an executive function. The inventive systems and methods may use a second set of data received by sensors and entered by a user to determine baseline user data. The inventive systems and methods may use user lifestyle data and determined baseline user data to determine performance potential for the user for an executive function (e.g., activity, job, etc.) which involves pressuring (e.g., stressing, exerting, etc.) the user. The inventive systems and methods may use a third set of data received by the sensors and entered by the user during the task to determine pressurized user data. The inventive systems and methods may use the determined baseline user data, the determined performance potential, and/or the determined pressurized user data to determine a user’s executive function ability. The present invention may reduce computational resources needed to assess a potential user’s fitness for a particular task.

The inventive systems and methods (hereinafter sometimes referred to more simply as “system” or “method”) described herein quantify team performance capacity. Specifically, the inventive systems and methods may track internal, external, environmental, etc. data about team members over time. For example, the inventive systems and methods may track sleep patterns for each or some team members. As another example, the inventive systems and methods may track the difference between an alarm set and an actual wake up time for each or some team members. As another example, the inventive systems and methods may track heart rates for each or some team members. As another example, the inventive systems and methods may track weather patterns associated with locations associated with each or some team members. The inventive systems and methods may determine if a team member is more toxicity than usual (e.g., less sleep, more time in between an alarm going off and getting up, higher heart rates, colder weather, etc.) or less toxicity than usual (detoxicity) (e.g., more sleep, less time in between an alarm going off and getting up, lower heart rates, consistent and/or warm weather, etc.). The inventive systems and methods may use a team member’s role to determine a weight to assign to an associated toxicity/detoxicity. For example, if a team member has a role of CEO, the team member’s toxicity/detoxicity may be weighted more than other team members. As another example, the more hours a team member works, the more weight their toxicity/detoxicity may be given. As another example, the higher the percentage of the total team salary an individual’s salary accounts for, the more weight their toxicity/detoxicity may be given. Each team member’s weighted toxicity/detoxicity may be used to compute a value representing how likely the team is to suffer from burnout. The present invention may reduce computational resources needed to for a team to achieve a goal by allowing a team to identify and address burnout before it affects the team.

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth herein in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

### Conceptual Architecture

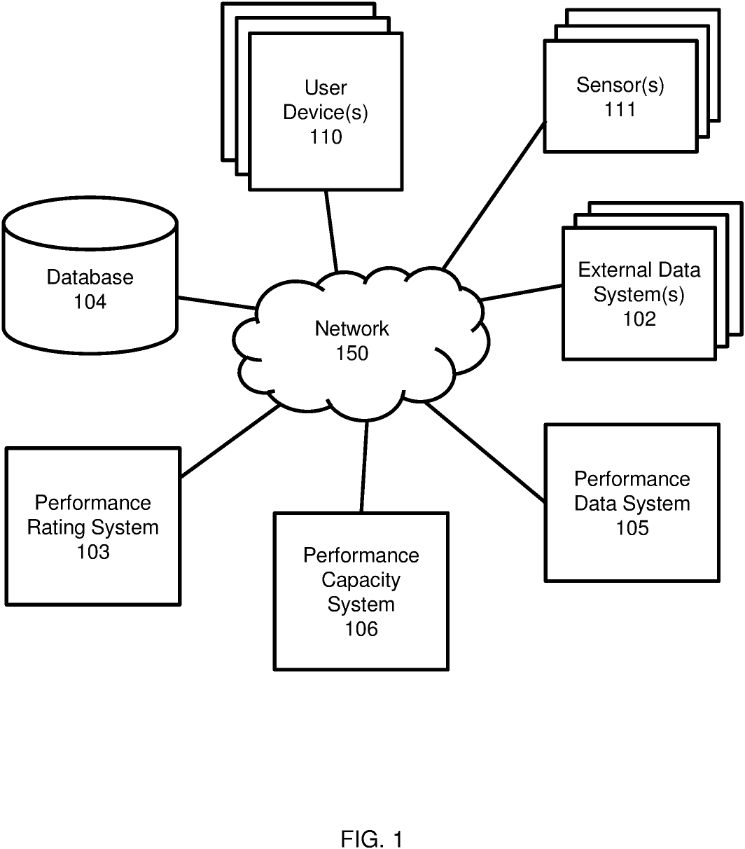

FIG. 1 illustrates an exemplary embodiment of a system for evaluating pressure performance and/or quantifying team performance capacity according to one embodiment. The system comprises, mental assessment system(s) 101, one or more external data system(s) 102, a performance rating system 103, a database 104, a performance data system 105, performance capacity system 106, one or more user device(s) 110, one or more sensors 111, and a network 150 over which the various systems communicate and interact. The various components described herein are exemplary and for illustration purposes only and any combination or subcombination of the various components may be used as would be apparent to one of ordinary skill in the art. The system may be reorganized or consolidated, as understood by a person of ordinary skill in the art, to perform the same tasks on one or more other servers or computing devices without departing from the scope of the invention.

The mental assessment system(s) 101 may comprise one or more computing devices. The mental assessment system(s) 101 may pull data from internal or external mental assessment databases, via the network 150, and return a score to the user device(s) 110 via the network 150.

The external data system(s) 102 may comprise one or more computing devices. The external data system(s) 102 may pull data from external databases. The external data systems(s) 102 may scrape data from external websites. The external data system(s) 102 may retrieve data subject to change or be dynamically updated, such as weather data, sunrise data, sunset data, etc. The external data system(s) 102 may retrieve industry standards (e.g., best practice guidance, etc.), such as, for example, updated blood pressure guidelines, updated sleep recommendations, updated calorie consumption recommendations, etc. The external data system(s) 102 may provide information, such as dynamic data, updated guidance etc., to the performance rating system 103 via the network 150.

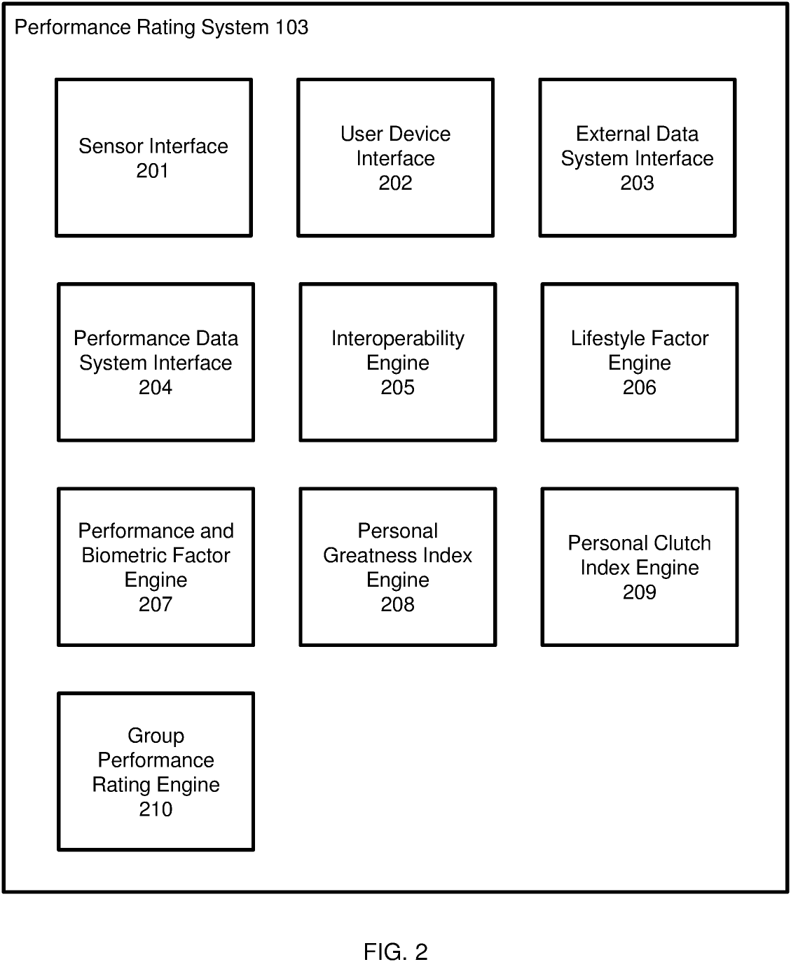

The performance rating system 103 may receive information from the user device(s) 110 and sensor(s) 111 associated with a user, via the network 150, and return a score to the user device(s) 110 via the network 150. The performance rating system 103 may receive data from one or more of the external data system(s) 102, the database 104, and a performance data system 105, via the network 150, and use the received data to compute the score returned to the user device(s) 110. The performance rating system 103 will be described in greater detail in FIG. 2.

The database 104 may store data accessible by the components of the system, such as the mental assessment system(s) 101, the performance rating system 103, the user device(s) 110, the sensor(s) 111, the external data system(s) 102, and the performance data system 105, via the network 150. Some components, such as the sensor(s) 111, may write and/or update fields in the database 104. Some components, such as the mental assessment system(s) 101, the performance rating system 103, the user device(s) 110, the external data system(s) 102, and the performance data system 105, may read, write, delete, and/or update fields in the database 104. Data stored in the database 104 may be associated with a user, a group of users containing a common trait, etc.

The performance data system 105 may compute data related to performance (e.g. time to complete, accuracy, etc.) under baseline and stress conditions. The computed data may comprise expected stress condition performances for particular baseline conditions. The computed data may be based on aggregating data from prior use. The computed data may be based on historical data of users with one or more similar characteristics of a current user. The performance data system 105 may update data based on new information from use of the performance rating system 103. The computed data may be for evaluating certain capabilities and/or executive functions, such as planning, etc. The computed data may comprise one or more quantifiable performance results, such as a time to complete, a success, and a relation to physiological data, etc. The computed data may comprise a goal, such as getting a heart rate up to a target, such as a threshold to simulate a “fight or flight” response (such as, for example, 160 beats per minutes), and then performing the task (e.g., activity, job, etc.).

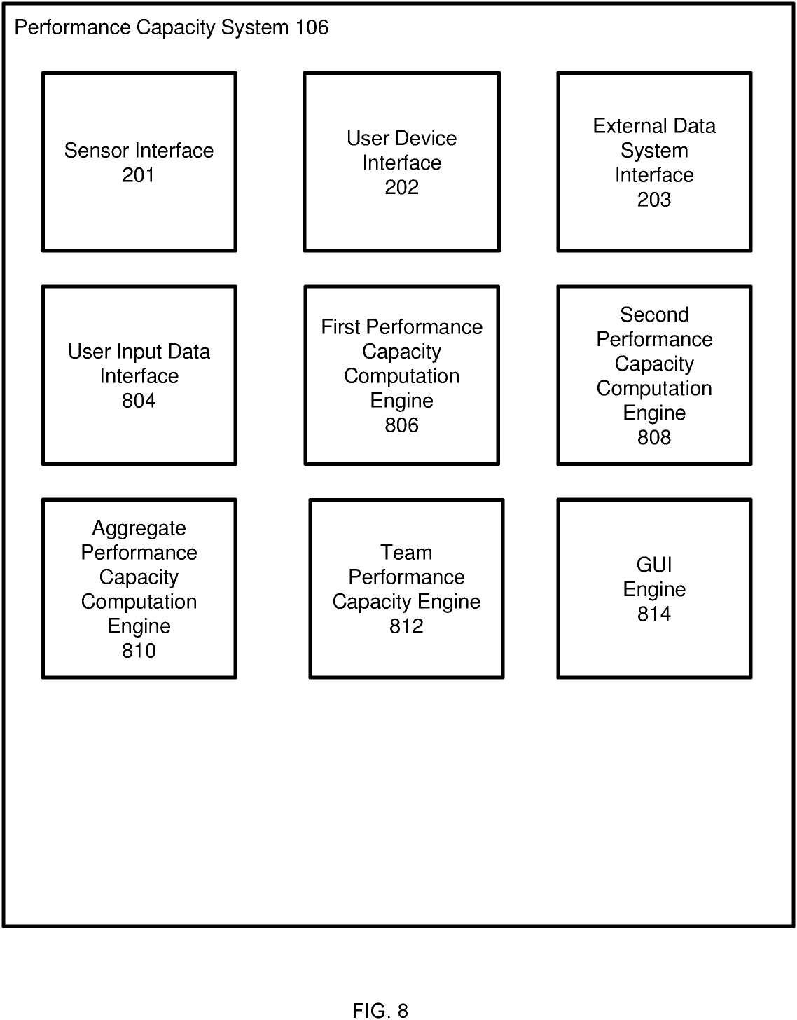

The performance capacity system 106 may receive information from the user device(s) 110 and sensor(s) 111 associated with a user, via the network 150, and return a score to the user device(s) 110 via the network 150. The performance capacity system 106 may receive data from one or more of the external data system(s) 102, the performance rating system 103, the database 104, and the performance data system 105, via the network 150, and use the received data to compute the score returned to the user device(s) 110. The performance capacity system 106 will be described in greater detail in FIG. 8.

The user device(s) 110 may comprise an application in communication with the performance rating system 103 via the network 150. The user device(s) 110 may transmit input manually entered by a user to the performance rating system 103. Although shown as communicating via the network 150, in an alternate embodiment, the sensor(s) 111 may communicate sensed data to the user device(s) 110, which then may transmit the sensed data to the performance rating system 103 via the network 150. The sensor(s) 111 may be attached to a user, such as a wearable smart device, a probe, a monitor, a blood pressure cuff, etc. The sensor(s) 111 may detect physiological information about a user. The sensor(s) 111 may transmit the detected information to the performance rating system 103 via the network 150. The sensor(s) 111 may transmit the detected information to the user device(s) 110 via the network 150. The sensor(s) 111 may transmit the detected information directly to the user device(s) 110.

The user device(s) 110 may include, generally, a computer or computing device including functionality for communicating (e.g., remotely) over a network 150. Data may be collected from user devices 110, and data requests may be initiated from each user device 110. User device(s) 110 may be a server, a desktop computer, a laptop computer, personal digital assistant (PDA), an in- or out-of-car navigation system, a smart phone or other cellular or mobile phone, or mobile gaming device, among other suitable computing devices. User devices 110 may execute one or more applications, such as a web browser (e.g., Microsoft Windows Internet Explorer, Mozilla Firefox, Apple Safari, Google Chrome, and Opera, etc.), or a dedicated application to submit user data, or to make prediction queries over a network 150.

In particular embodiments, each user device 110 may be an electronic device including hardware, software, or embedded logic components or a combination of two or more such components and capable of carrying out the appropriate functions implemented or supported by the user device 110. For example and without limitation, a user device 110 may be a desktop computer system, a notebook computer system, a netbook computer system, a handheld electronic device, or a mobile telephone. The present disclosure contemplates any user device 110. A user device 110 may enable a network user at the user device 110 to access network 150. A user device 110 may enable its user to communicate with other users at other user devices 110.

A user device 110 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user device 110 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 110 one or more Hyper Text Markup Language (HTML) files responsive to the HTTP request. The user device 110 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 110 may also include an application that is loaded onto the user device 110. The application obtains data from the network 150 and displays it to the user within the application interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing systems may perform at different times or at different locations one or more steps of one or more methods described or illustrated herein, where appropriate.

Network cloud 150 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 421 or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to client/user devices or other devices in response to HTTP or other requests from client devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiment, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

FIG. 2 illustrates an exemplary embodiment of the performance rating system 103. The executive function performance rating system 103 may generate executive function performance scores for individuals based on a combination of an individual’s mental factors, lifestyle factors and an individual’s response to stressors. The exemplary performance rating system 103 may comprise a sensor interface 201, a user device interface 202, an external data system interface 203, a performance data system interface 204, an interoperability engine 205, a lifestyle factor engine 206, a performance and biometric factor engine 207, a personal greatness index engine 208, a personal clutch index engine 209, mental assessment engine 211, and a group performance rating engine 210. The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met. As used below, sending or receiving data may be the same as sending or receiving one or more signals indicative of the sent or received data.

The sensor interface 201 may receive data from the network 150 in FIG. 1 originating from the sensor(s) 111 in FIG. 1 and prepare the received data in a manner suitable for consumption by other elements of the performance rating system 103. For example, the sensor interface 201 may receive data originating from a smartwatch and prepare the received data in a manner fit for the lifestyle factor engine 206. As another example, the sensor interface 201 may receive data originating from a probe attached to a user’s skin and prepare the received data in a manner fit for the biometric factor engine 207. As another example, the sensor interface 201 may receive data originating from a hormone test, such as a cortisol test, and prepare the received data in a manner fit for the personal greatness index engine 208. As another example, the sensor interface 201 may receive data originating from a hormone test, such as a testosterone test, and prepare the received data in a manner fit for the personal clutch index engine 209. Although several examples were shown, it is contemplated that the sensor interface 201 may receive data from any sensor(s) 111 and prepare the received data in a manner suitable for consumption by any of the elements of the performance rating system 103.

The user device interface 202 may receive data from the network 150 in FIG. 1 originating from the user device(s) 110 in FIG. 1 and prepare the received data in a manner suitable for consumption by other elements of the performance rating system 103. The user device interface 202 may receive data from other elements of the performance rating system 103 and prepare the received data in a manner suitable from transmission across the network 150 and ultimate consumption by the user device(s) 110. The user device(s) 110 may receive input from a user and transmit received input via the network 150 to the user device(s) 110, wherein the user device interface 202 prepares the received input for consumption by any of the elements of the performance rating system 103.

The external data system interface 203 may receive data from the network 150 in FIG. 1 originating from the external data system(s) 102 in FIG. 1 and prepare the received data in a manner suitable for consumption by other elements of the performance rating system 103. The external data system interface 203 may pull data from the external data system(s) 102. The external data system interface 203 may scrape data from the external data system(s) 102. The external data system interface 203 may retrieve data subject to change or be dynamically updated, such as weather data, sunrise data, sunset data, etc. The external data system interface 203 may retrieve industry standards (e.g., best practice guidance, etc.), such as, for example, updated blood pressure guidelines, updated sleep recommendations, updated calorie consumption recommendations, etc. The external data system interface 203 may provide information, such as dynamic data, updated guidance etc., to the other elements of the performance rating system 103.

The performance data system interface 204 may retrieve data from the network 150 in FIG. 1 originating from the performance data system 105 in FIG. 1 and prepare the received data in a manner suitable for consumption by other elements of the performance rating system 103. The performance data system interface 204 may receive data from other elements of the performance rating system 103 and prepare the received data in a manner suitable from transmission across the network 150 and ultimate consumption by the performance data system 105. The performance data system interface 204 may obtain data related to performance (e.g. time to complete, accuracy, etc.) under baseline and stress conditions. The obtained data may comprise expected stress condition performances for particular baseline conditions. The obtained data may be based on aggregating data from prior use. The obtained data may be based on historical data of users with one or more similar characteristics of a current user. The performance data system interface 204 may transmit data to the performance rating system 103 to update data stored by the performance data system 105. The performance data system interface 204 may obtain data for evaluating certain capabilities and/or executive functions, such as planning, etc. The obtained data may comprise one or more quantifiable performance results, such as a time to complete, a success, and a relation to physiological data, etc. The obtained data may comprise a goal, such as getting a heart rate up to a target, such as a threshold to simulate a “fight or flight” response (such as, for example, 160 beats per minutes), and then performing the task (e.g., activity, job, etc.).

The interoperability engine 205 may convert data from various different systems and/or elements into standard formats for further analysis and/or processing. For example, the interoperability engine 205 may convert data to and/or from the metric system. As another example, the interoperability engine 205 may convert timestamps into a standard time, such as the Coordinated Universal Time (UTC).

The lifestyle factor engine 206 may be operable to convert physiology data from sensors and engagement data from user devices into lifestyle factor metrics. Lifestyle factor metrics may comprise at least one of a punctuality factor, sleep factor, a diet factor, an exercise factor, a bodily waste factor, and a mental status factor. In one aspect, lifestyle factor metrics may comprise or be categorized as at least one of active lifestyle factors and/or passive lifestyle factors. Active lifestyle factors may comprise factors which an individual may be aware of and/or knowingly adjust. Active lifestyle factors may comprise at least one of diet and exercise. Passive lifestyle factors may comprise factors which an individual may be unaware of and/or unable to knowingly adjust. Passive lifestyle factors may comprise at least one of REM sleep, sleep time/duration relative to sunset and/or sunrise, punctuality and waste excretion. In one aspect, the lifestyle factor data may be categorized as contributing to toxicity generation or toxicity reduction (or recovery). In one aspect, lifestyle factor data may be combined to generate a net toxicity (or recovery) metric.

The performance and biometric factor engine 207 may convert physiology data from sensors and performance data from the performance data system 105 in FIG. 1 into performance and biometric factor data. The performance and biometric factor data may comprise measurements of executive function change under stress. The performance and biometric factor data may comprise executive function data obtained during baseline activity and data obtained during at least one of mental stress activity and/or physical stress activity.

The personal greatness index (PGI) engine 208 may be operable to compute a PGI score based on at least the lifestyle factor data. The PGI score may be a novel reflection of an individual’s executive function performance potential based on lifestyle characteristics. In general, accumulated toxicity (net or total toxicity over time) may be associated with reduced synapse frequency, while reduced toxicity (net or total reduction of toxicity over time or recovery over time) may be associated with maintaining a higher level of cognitive performance potential and/or improving cognitive performance potential with respect to cognitive performance levels associated with higher toxicity metrics (or lower recovery metrics). The PGI score may provide an indication of an individual’s executive function performance potential as determined based on toxicity and/or recovery data over time. For example, poor sleep hygiene over a period of one or more days may lead to reduced cognitive performance for an individual. This poor sleep hygiene would contribute to an increase in the toxicity metric which would adversely affect an individual’s PGI.

The lutch index engine 209 may compute a clutch score based on the executive function performance and biometric factor data. The Clutch index engine 209 may optionally incorporate a user’s PGI into the calculation to normalize the score to account for varying lifestyle impacts.

The group executive function performance rating engine 210 may rank clutch data and/or performance and/or biometric factor data for a group of individuals (e.g. team, demographic, etc.). The group performance rating engine 210 may compute a group clutch score based on individual ranking amongst the group. The group executive function performance rating engine 210 may optionally incorporate the users’ PGI into the calculation to normalize the score to account for varying lifestyle impacts of the users.

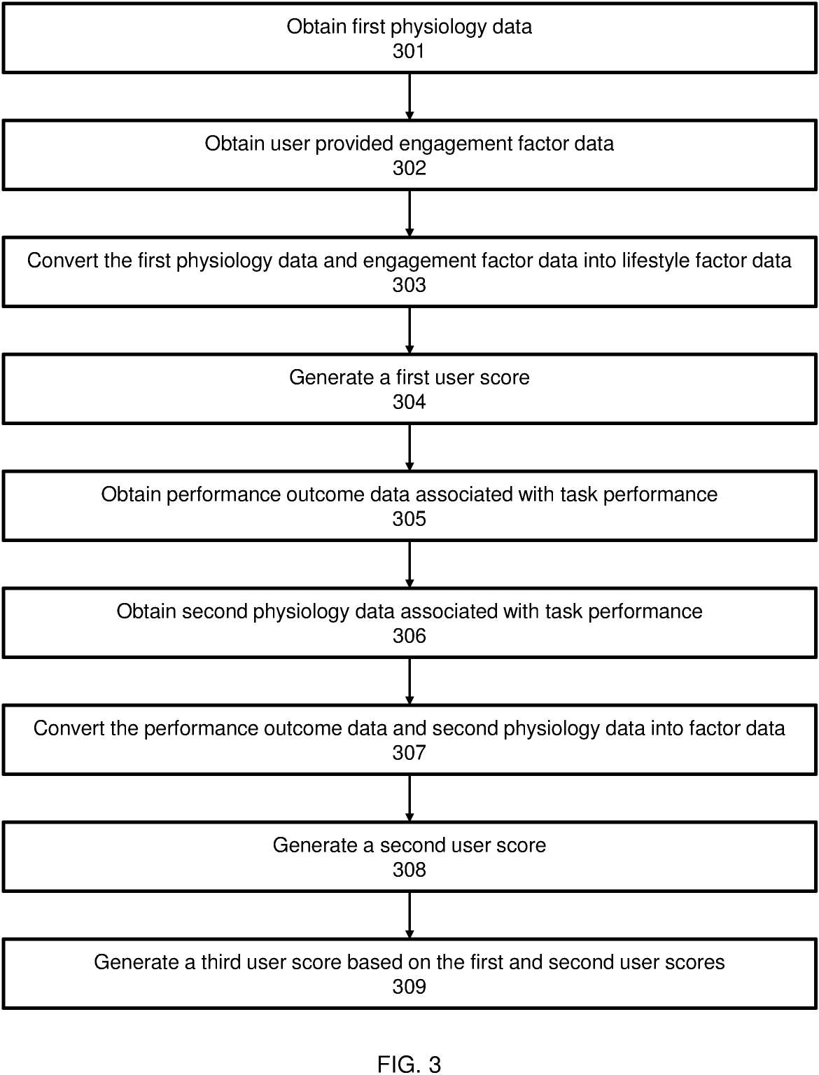

FIG. 3 illustrates an exemplary process for evaluating executive function under pressure according to one embodiment of the invention. In embodiments, the method steps or techniques depicted and described herein can be performed in a processor of the performance rating system 103 in FIG. 1, the method steps being encoded as processor-executable instructions in a non-transitory memory of the performance rating system 103. The techniques of FIG. 3 may be implemented in an operating system kernel, in a separate user process, in a library package bound into network applications, on a specially constructed machine, on an application-specific integrated circuit (ASIC), or a field programmable gate array (FPGA). The process may comprise additional steps, fewer steps, and/or a different order of steps without departing from the scope of the invention as would be apparent to one of ordinary skill in the art.

At step 300, baseline mental status data may be obtained. The baseline mental status data maybe collected by user answering up to 300 questions, selecting 1 to 10 for each question

At step 301, first physiology data may be obtained. The first physiology data may be detected by at least one first user worn sensor. The first physiology data may be associated with a user. The first physiology data may comprise at least one of heart rate (HR) data, heart rate variability (HRV) data, sleep time data, wake time data, sleep duration data, rapid eye movement (REM) sleep duration data, deep sleep duration data, and calories burned data.

At step 302, user provided engagement factor data may be obtained. The user provided engagement factor data may originate from at least one first user device. The user may manually input some or all of the user provided engagement factor data. The engagement factor data may comprise user input associated with at least one of punctuality, sleep, diet, waste excretion, exercise, and mental status.

At step 303, the first physiology data and engagement factor data may be converted into lifestyle factor data. A processor may convert the first physiology data and engagement factor data into lifestyle factor data. The lifestyle factor data may have a standard format. The lifestyle factor data may comprise a plurality of components. Each of the plurality of components may be associated with at least one of sleep, diet, exercise, waste excretion, and mental status. The lifestyle factor data may comprise at least one of active lifestyle factor data and passive lifestyle factor data. Each lifestyle factor may be categorized as associated with toxicity generation or toxicity reduction. A net toxicity metric may be computed from the lifestyle factor data.

At step 304, a first user score may be generated. A processor may generate the first user score. The first user score may be associated with the lifestyle factor data. The first user score may provide an indication of user executive function performance potential. The executive function performance potential may be determined as a function of at least one of toxicity accumulation over time and toxicity reduction over time.

At step 305, executive function performance outcome data associated with cognitive task performance may be obtained. A processor may obtain the executive function performance outcome data. The executive function performance outcome data may be associated with a cognitive task performed by the user under at least one condition. The at least one condition may comprise at least one of a baseline condition, a mental stimulation or mental stress condition, and a physical exertion condition. The mental stress condition may be invoked by applying at least one external stimuli to be sensed by the user. The physical exertion condition may be invoked by requiring the user to perform a physical activity during and/or prior to performing the task. The physical exertion condition may comprise requiring the user to achieve certain physiological criteria prior to or during performing the task.

At step 306, second physiology data associated with task performance may be obtained. The second physiology data may be detected by the at least one first user worn sensor. The second physiology data may be associated with the task performed by the user under the at least one condition. The second physiology data may comprise at least one of heart rate (HR) data, heart rate variability (HRV) data, blood pressure data, posture data, and hormonal data. Biometric change data may be computed. The biometric change data indicating a change in the second physiology data associated with at least one of the mental stress condition and the physical stress condition as compared to the first physiology data associated with the baseline condition.

At step 307, the performance outcome data and second physiology data may be converted into factor data. A processor may convert the performance outcome data and second physiology data into factor data. The factor data may have a standard format. The performance outcome data may be converted into performance factor data having a standard format. The second physiology data may be converted into biometric factor data having a standard format.

At step 308, a second user score may be generated. A processor may generate the second user score. The second user score may be associated with the biometric factor data and performance factor data. The second user score may indicate at least one of an average biometric change associated with a plurality of conditions and an average performance change associated with a plurality of conditions.

At step 309, a third user score may be generated based on the first and second user scores. A processor may generate the third user score. The third user score may comprise an adjustment of the second user score based on a ratio of the first user score relative to a first user score threshold target. The first user score threshold target may be indicative of a threshold below which performance potential is reduced.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments).

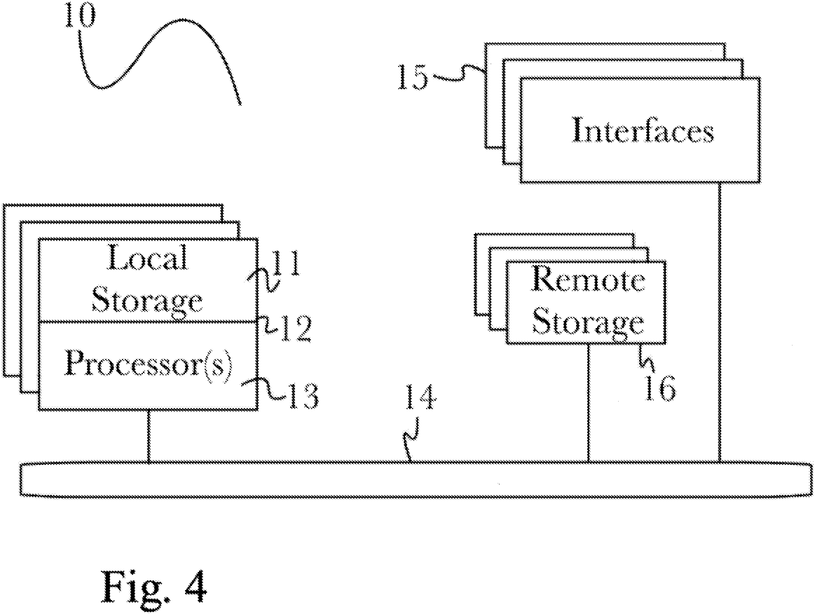

Any of the above mentioned systems, units, modules, engines, controllers, interfaces, components or the like may be and/or comprise hardware and/or software as described herein. For example, the performance rating system 103 and subcomponents thereof may be and/or comprise computing hardware and/or software as described herein in association with FIGS. 4-7. Furthermore, any of the above mentioned systems, units, modules, engines, controllers, interfaces, components or the like may use and/or comprise an application programming interface (API) for communicating with other systems units, modules, engines, controllers, interfaces, components, or the like for obtaining and/or providing data or information.

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine- readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

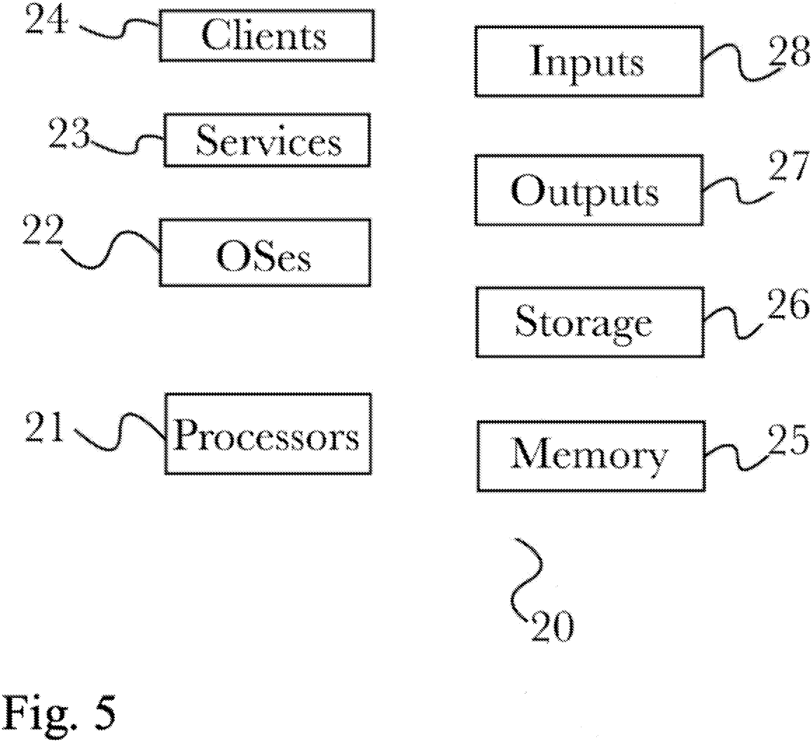

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

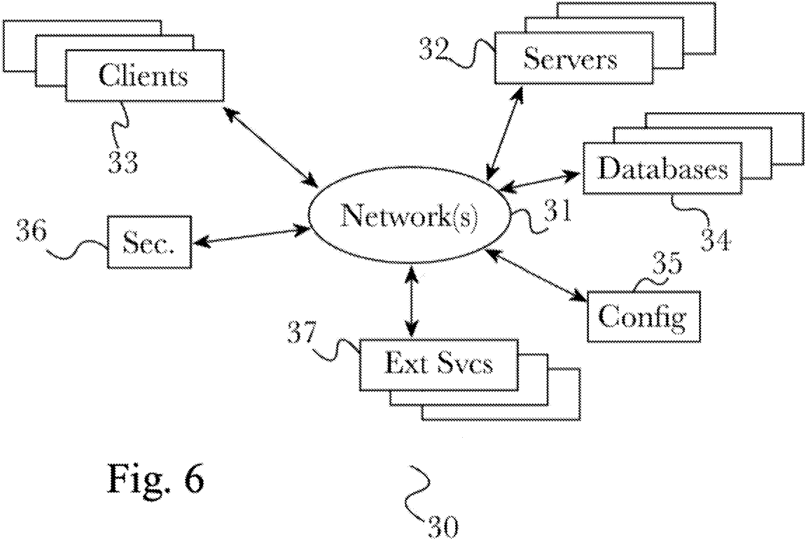

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise’s or user’s premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

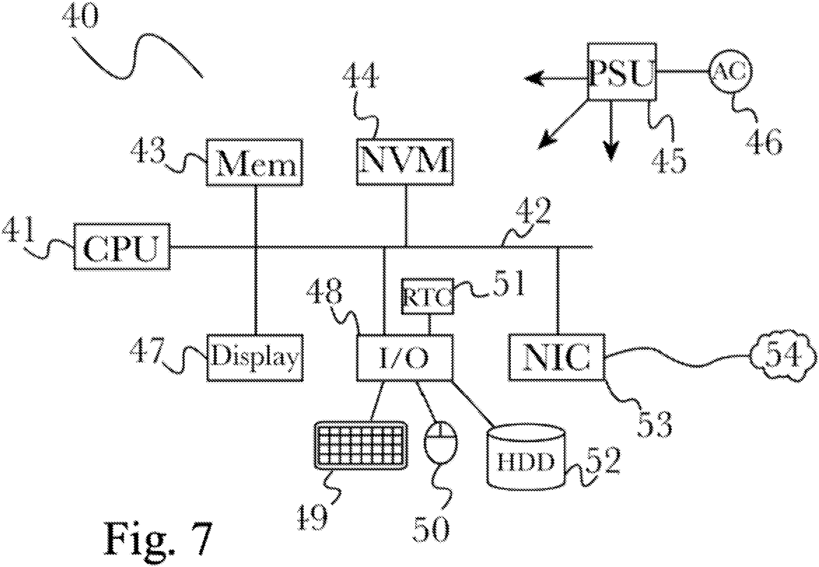

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Quantifying Team Performance Capacity

FIG. 8 illustrates an exemplary embodiment of the performance capacity system 106. The performance capacity system 106 may generate performance capacity scores for teams based on a combination of team member’s active lifestyle factor data, passive lifestyle factor data, physiological data input, external conditions data, and computing emotional intelligence (EI) score data. The exemplary performance capacity system 106 may comprise a sensor interface 201, a user device interface 202, an external data system interface 203, a user input data interface 804, a first performance capacity computation engine 806, a second performance capacity computation engine 808, an aggregate performance capacity computation engine 810, a team performance capacity engine 812, and a graphical user interface (GUI) engine 814. The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met. As used below, sending or receiving data may be the same as sending or receiving one or more signals indicative of the sent or received data.

The sensor interface 201 may receive data from the network 150 in FIG. 1 originating from the sensor(s) 111 in FIG. 1 and prepare the received data in a manner suitable for consumption by other elements of the performance capacity system 106. For example, the sensor interface 201 may receive data, such as bio data (such as heart rate, skin temperature, etc.), originating from a smartwatch and prepare the received data in a manner fit for other components of the performance capacity system 106. As another example, the sensor interface 201 may receive data originating from a probe attached to a user’s skin and prepare the received data in a manner fit for other components of the performance capacity system 106. As another example, the sensor interface 201 may receive data originating from a hormone test, such as a cortisol test, and prepare the received data in a manner fit for other components of the performance capacity system 106. Although several examples were shown, it is contemplated that the sensor interface 201 may receive data from any sensor(s) 111 and prepare the received data in a manner suitable for consumption by any of the elements of the performance capacity system 106.

The user device interface 202 may receive data from the network 150 in FIG. 1 originating from the user device(s) 110 in FIG. 1 and prepare the received data in a manner suitable for consumption by other elements of the performance capacity system 106. The user device interface 202 may receive data from other elements of the performance capacity system 106 and prepare the received data in a manner suitable from transmission across the network 150 and ultimate consumption by the user device(s) 110. The user device(s) 110 may receive input from a user and transmit received input via the network 150 to the user device(s) 110, wherein the user device interface 202 prepares the received input for consumption by any of the elements of the performance capacity system 106. The user device interface 202 may receive data, such as geo location information, device alarm information, accelerometer information, etc.

The external data system interface 203 may receive data from the network 150 in FIG. 1 originating from the external data system(s) 102 in FIG. 1 and prepare the received data in a manner suitable for consumption by other elements of the performance capacity system 106. The external data system interface 203 may pull data from the external data system(s) 102. The external data system interface 203 may scrape data from the external data system(s) 102. The external data system interface 203 may retrieve data subject to change or be dynamically updated, such as weather data, sunrise data, sunset data, etc. The external data system interface 203 may retrieve industry standards (e.g., best practice guidance, etc.), such as, for example, updated blood pressure guidelines, updated sleep recommendations, updated calorie consumption recommendations, etc. The external data system interface 203 may provide information, such as dynamic data, updated guidance etc., to the other elements of the performance capacity system 106.

The user input data interface 804 may receive data from the network 150 in FIG. 1 originating from the user device(s) 110 in FIG. 1 and prepare the received data in a manner suitable for consumption by other elements of the performance capacity system 106. The user input data interface 804 may receive data from other elements of the performance capacity system 106 and prepare the received data in a manner suitable from transmission across the network 150 and ultimate consumption by the user device(s) 110 for display on a screen. The user device(s) 110 may receive input from a user and transmit received input via the network 150 to the user device(s) 110, wherein the user input data interface 804 prepares the received input for consumption by any of the elements of the performance capacity system 106. The user input data interface 804 may cause a questionnaire (e.g., form, poll, etc.) to be displayed on the user device(s) 110. The user input data interface 804 may receive input from the displayed questionnaire. The user input data interface 804 may calculate an emotional intelligence (EI) for a user based on the received input. The user input data interface 804 may cause the calculated EI to be displayed on the user device(s) 110.

The first performance capacity computation engine 806 may compute a first performance capacity score. The first performance capacity score may be computed in part based on a relationship between data received from a user device 110 in FIG. 1 and/or sensor(s) 111 in FIG. 1 and data pulled from a database via an Application Programming Interface (API). For example, the first performance capacity score may be based in part based on a relationship between detected bedtime data and retrieved sunset data for an associated location and date. As another example, the first performance capacity score may be based in part on a relationship between geo location data associated with a user device 110 and a weather forecast retrieved for an associated location.

The second performance capacity computation engine 808 may compute a second performance capacity score. The second performance capacity score may be based on data received from user device(s) 110 in FIG. 1 and/or sensor(s) 111 in FIG. 1. For example, the second performance capacity score may be based on at least one of an exercise measure, a resting heart rate measure, a sleep quality measure, etc. The second performance capacity score may be computed in part based on a relationship between data received from user device(s) 110 and/or sensor(s) 111 and data pulled from a database via an Application Programming Interface (API). For example, the second performance capacity score may be based at least in part on a relationship between dinner time and sunset. For example, the second performance capacity score may be based at least in part on a relationship between bedtime data and sunset data.

The aggregate performance capacity computation engine 810 may track performance capacity data for a user over time to create historical data for the user. Historical performance capacity data may be used to create an average historical performance capacity data over a time period, such as, for example, the last 5 days. The average historical performance capacity data may be used to compare against recent entries, such as a most recent entry or the two most recent entries, etc. When comparing average historical performance capacity data with recent entries, the more recent a recent entry is, the more weight the recent entry may have in the comparison.

The team performance capacity engine 812 may consider performance capacity scores for individual team members to determine a capacity score for a team. Each team member’s performance capacity score may be weighted based on the associated team member’s role in the team. Weighting performance capacity scores may involve weighting performance capacity scores based on job titles (e.g., positions, ranks, etc.), hours worked, relative salary, etc. For example, a performance capacity score associated with a chief executive officer (CEO) may have more weight than a performance capacity score associated with a happiness engineer. As another example, a performance capacity score associated with a worker that has worked 600 hours the previous quarter may have more weight than a performance capacity score associated with a worker that has worked 450 hours the previous quarter. The relative salary may be a ratio representing an associated user’s salary compared to the total team salary. The weight of a performance capacity score associated with a team member may be or be based on the relative salary of the team member.

The graphical user interface (GUI) engine 814 may prepare data for presentation on user device(s) 110. For example, the GUI engine 814 may prepare performance capacity data over a period of time (e.g., week, month, etc.) into a chart or graph for easy digestion by a user. The GUI engine 814 may display various aspects of a team member’s recent performance capacity data, such as, for example, sleep time compared to sunset time, as compared to reference data. The GUI engine 814 may display a computed score for the team members recent performance capacity data based on the various aspects as compared to reference data. Reference data may comprise other team member’s recent and/or historical performance capacity data. Reference data may comprise historical performance capacity data for the team member. The GUI engine 814 may highlight particularly problematic and/or exemplary aspects of the team member’s performance capacity data. The GUI engine 814 may make suggestions for improving a team member’s performance capacity data.

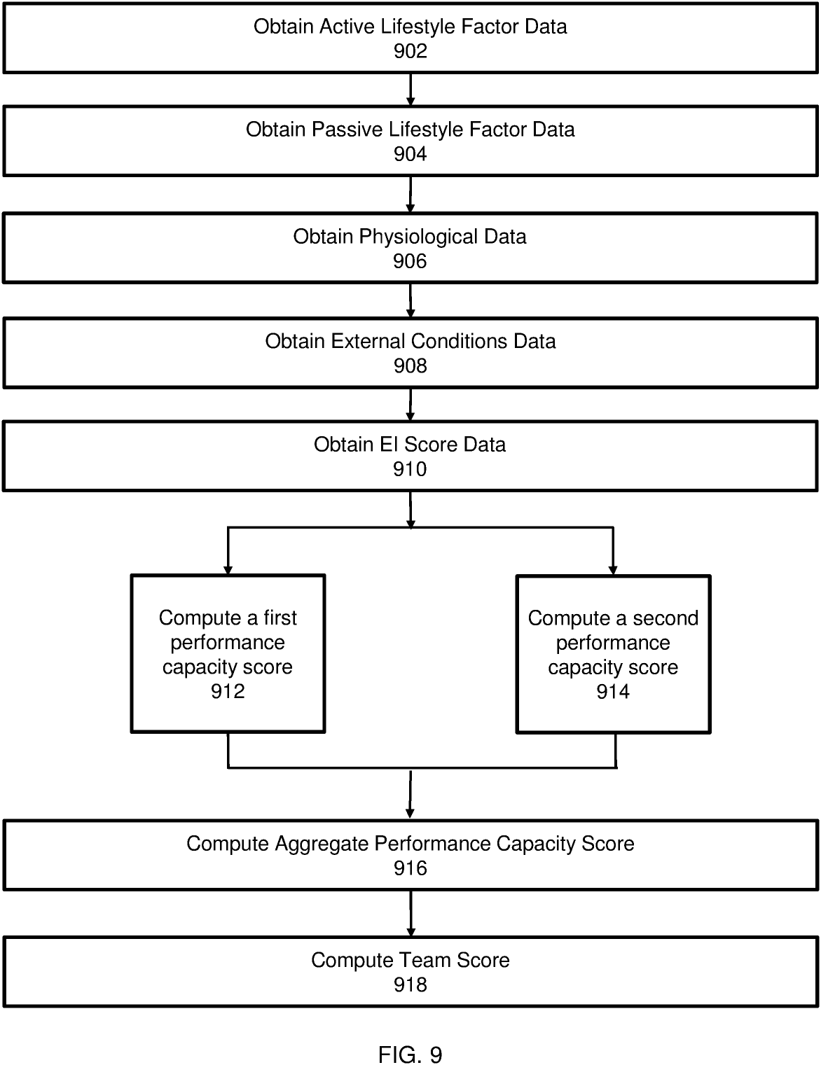

FIG. 9 illustrates an exemplary process for quantifying team performance capacity according to one embodiment of the invention. In embodiments, the method steps or techniques depicted and described herein can be performed in a processor of the performance capacity system 106 in FIG. 1, the method steps being encoded as processor-executable instructions in a non-transitory memory of the performance capacity system 106. The techniques of FIG. 9 may be implemented in an operating system kernel, in a separate user process, in a library package bound into network applications, on a specially constructed machine, on an application-specific integrated circuit (ASIC), or a field programmable gate array (FPGA). The process may comprise additional steps, fewer steps, and/or a different order of steps without departing from the scope of the invention as would be apparent to one of ordinary skill in the art.

At step 902, active lifestyle factor data associated with at least one user in the team may be obtained via at least one of a first user worn sensor and a first user device. Active lifestyle factor data may comprise at least one of exercise data, dinnertime data, bedtime data, and punctuality data. Exercise data may comprise at least one of exercise duration data and exercise intensity data. Punctuality data may comprise at least one of calendar event data and user performance of calendar event data.

At step 904, passive lifestyle factor data associated with at least one user in the team may be obtained via the first user worn sensor. Passive lifestyle data may comprise at least one of resting heart rate data, REM sleep duration data, and deep sleep duration data.

At step 906, physiological data input by at least one user in a team may be obtained via the first user device. User input physiological data may comprise food intake data.

At step 908, external conditions data may be obtained via an application programming interface (API). External conditions data may comprise at least one of outdoor temperature data, dew point data, outdoor temperature data, atmospheric pressure data, and Earth to moon distance data.

At step 910, emotional intelligence (EI) score data may be computed based on EI input data. The EI input data may be input on the first user device by at least one user in the team. Remote access may be provided to users over a network so any one of the users can update information about the physiological data and the EI input data. Any one of the users may provide the update information in a non-standardized format depending on at least one of the hardware and platform used by the any one of the users. The obtained passive lifestyle factor data, the physiological data, the API data, the EI score data, and the non-standardized update information may be converted into a standardized format.

At step 912, a first performance capacity score for at least one user in the team may be computed based on the active lifestyle data, the external data, and user input physiological data. The first performance capacity score may be computed in part based on a relationship between the active lifestyle data and the API data. The first performance capacity score may be based in part based on a relationship between bedtime data and sunset data.

At step 914, a second performance capacity score for at least one user in the team may be computed based on active lifestyle data, passive lifestyle data, and API data. The second performance capacity score may be based on at least one of an exercise measure, a resting heart rate measure, a relationship between dinner time and sunset, a sleep quality measure, and a relationship between bedtime data and sunset data.

At step 916, an aggregated performance capacity score for at least one user on the team may be computed based on the first performance capacity score and the second performance capacity score. The first performance capacity score may be from a first user at a first time and the second performance capacity score may be from a second user at a first time. The first performance capacity score may be from a first user at a first time and the second performance capacity score may be from a first user at a second time. The first performance capacity score may be from a first user at a first time and the second performance capacity score may be from a second user at a second time.

At step 918, a team performance score may be computed based on individual role data. The role data may indicate an individual’s contribution to the team. The role data may comprise at least one of a position, hours worked, and relative salary. The relative salary may be a ratio representing an associated user’s salary compared to the total team salary. A message containing an aggregated performance capacity score and the team performance score may be automatically generated whenever new lifestyle factor data, passive lifestyle data, physiological data, external condition data, EI input data, or non-standardized update data is obtained. The message may be transmitted to a plurality of users over the computer network in near real time, so that the plurality of users have near immediate access to at least one of an aggregated performance capacity score and the team performance score. The team performance score may be further based on a third performance capacity score associated with another user.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for evaluating pressure performance and/or quantifying team performance capacity through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A computer implemented method for quantifying performance capacity associated with a team, the computer implemented method comprising:
obtaining, via at least one of a first user worn sensor and a first user device, active lifestyle factor data associated with at least one user in the team;
obtaining, via the first user worn sensor, passive lifestyle factor data associated with at least one user in the team;
obtaining, via the first user device, physiological data input by at least one user in a team;
obtaining, via an application programming interface (API), external conditions data;
computing emotional intelligence (EI) score data based on EI input data;
computing a first performance capacity score for at least one user in the team, based on the active lifestyle data, the external data, and user input physiological data;
computing a second performance capacity score for at least one user in the team based on active lifestyle data, passive lifestyle data, and API data;
computing an aggregated performance capacity score for at least one user on the team based on the first performance capacity score and the second performance capacity score; and
computing a team performance score based on individual role data.

2. The computer implemented method of claim 1, wherein active lifestyle factor data comprises at least one of exercise data, dinnertime data, bedtime data, and punctuality data.

3. The computer implemented method of claim 2, wherein exercise data comprises at least one of exercise duration data and exercise intensity data.

4. The computer implemented method of claim 2, wherein punctuality data comprises at least one of calendar event data and user performance of calendar event data.

5. The computer implemented method of claim 1, wherein passive lifestyle data comprises at least one of resting heart rate data, REM sleep duration data, and deep sleep duration data.

6. The computer implemented method of claim 1, wherein user input physiological data comprises food intake data.

7. The computer implemented method of claim 1, wherein external conditions data comprises at least one of outdoor temperature data, dew point data, outdoor temperature data, atmospheric pressure data, and Earth to moon distance data.

8. The computer implemented method of claim 1, wherein the EI input data is input on the first user device by at least one user in the team.

9. The computer implemented method of claim 1, further comprising providing remote access to users over a network so any one of the users can update information about the physiological data and the EI input data.

10. The computer implemented method of claim 9, wherein any one of the users provides the update information in a non-standardized format depending on at least one of the hardware and platform used by the any one of the users.

11. The computer implemented method of claim 10, further comprising converting the obtained passive lifestyle factor data, the physiological data, the API data, the EI score data, and the non-standardized update information into a standardized format.

12. The computer implemented method of claim 1, wherein the first performance capacity score is computed in part based on a relationship between the active lifestyle data and the API data.

13. The computer implemented method of claim 1, wherein the first performance capacity score is based in part based on a relationship between bedtime data and sunset data.

14. The computer implemented method of claim 1, wherein the second performance capacity score is based on at least one of an exercise measure, a resting heart rate measure, a relationship between dinner time and sunset, a sleep quality measure, and a relationship between bedtime data and sunset data.

15. The computer implemented method of claim 1, wherein the role data indicates an individual’s contribution to the team.

16. The computer implemented method of claim 1, wherein the role data comprises at least one of a position, hours worked, and relative salary.

17. The computer implemented method of claim 16, wherein the relative salary is a ratio representing an associated user’s salary compared to the total team salary.

18. The computer implemented method of claim 1, further comprising automatically generating a message containing an aggregated performance capacity score and the team performance score whenever new lifestyle factor data, passive lifestyle data, physiological data, external condition data, EI input data, or non-standardized update data is obtained.

19. The computer implemented method of claim 1, further comprising transmitting the message to a plurality of users over the computer network in near real time, so that the plurality of users have near immediate access to at least one of an aggregated performance capacity score and the team performance score.

20. The computer implemented method of claim 1, wherein the team performance score is further based on a third performance capacity score associated with another user.
